(19) United States
(12) Patent Application Publication
(10) Pub. No.: US 2023/0082473 A1
(43) Pub. Date: Mar. 16, 2023

(54) METHOD FOR REDUCING NUCLEIC ACID AND ADSORBING FILTER

(71) Applicant: KANEKA CORPORATION, Osaka-shi, Osaka (JP)

(72) Inventors: Fuminori KONOIKE, Takasago-shi (JP); Kazunobu MINAKUCHI, Takasago-shi (JP); Dai MURATA, Takasago-shi (JP); Kenta KISHI, Takasago-shi (JP); Masakatsu NISHIHACHIJO, Takasago-shi (JP)

(73) Assignee: KANEKA CORPORATION, Osaka-shi, Osaka (JP)

(21) Appl. No.: 17/801,715

(22) PCT Filed: Feb. 19, 2021
(86) PCT No.: PCT/JP2021/006411

§ 371 Date: Aug. 23, 2022

(30) Foreign Application Priority Data
Feb. 28, 2020 (JP) 2020-033132
Feb. 28, 2020 (JP) 2020-033216

(51) Int. Cl. B01J 20/04; C12N 1/08; C12N 7/00; C07K 1/22; B01D 39/16; B01D 39/20

(52) U.S. Cl. CPC B01J 20/043; C12N 1/08; B01J 20/041; C12N 7/00; C07K 1/22; B01D 39/1692; B01D 39/2031; B01D 2239/0407

(57) ABSTRACT

The objective of the present invention is to provide a method for effectively and easily reducing an amount of a specific impurity in a liquid. The method for reducing an amount of a nucleic acid in a liquid according to the present invention is characterized in comprising the steps of contacting the liquid with a water-insoluble magnesium compound to adsorb at least a part of the nucleic acid on the water-insoluble magnesium compound. Also, the objective of the present invention is to provide an adsorbing filter useful for purifying a useful substance, such as an antibody and an antibody-like molecule, used as a purification material for effectively removing an impurity with easily maintaining the yield of the target substance due to excellent adsorption ability to a nucleic acid and low adsorption ability to an antibody, an antibody-like molecule or the like. The adsorbing filter is characterized in comprising the layer comprising a water-insoluble magnesium compound.

### TECHNICAL FIELD

The present invention relates to a method for effectively and easily reducing an amount of a nucleic acid in a liquid due to excellent adsorption ability to a nucleic acid and low adsorption ability to an antibody, an antibody-like molecule or the like, and an adsorbing filter useful for purifying a useful substance, such as an antibody and an antibody-like molecule, used as an active ingredient of a bio-pharmaceutical product.

### BACKGROUND ART

A protein is mainly produced by a transformation method since a transformation method using a plasmid containing a gene encoding a target protein was put to practical use. For example, an antibody and an antibody-like molecule can be produced by a transformation method using a human gene. An antibody-like molecule consists of a part necessary for antigen recognition and cellular cytotoxicity among antibodies. A virus and a virus-like particle to be used as vaccine and a vector for gene therapy are generally produced by infecting a cultivated cell or a fertilized egg of a chicken with the virus and growing the virus and the virus-like particle in a culture fluid or a body fluid. A useful substance such as a protein and a virus is purified by first removing a solid such as a cell and a cell fragment from a culture fluid or the like with filtration and then carrying out chromatography or the like.

An example of an impurity other than a useful substance includes a misplaced material such as a protein, a nucleic acid and a lipid derived from a host and a living body. In particular, a nucleic acid may make filtration difficult by increasing viscosity of a liquid to deteriorate liquid permeability and decrease purification efficiency by fouling a chromatography carrier and a membrane in some cases. In addition, the nucleic acid derived from a certain kind of a host such as an immortalized cell must be completely removed, since such a nucleic acid involves the risk of the cancerization of a patient's cell.

A protein and a virus are also used for examination and diagnosis. For example, a reagent for examination and diagnosis to identify a virus is developed on the basis of a base sequence of the virus in addition to the use of an antigen-antibody reaction. Also, examination to detect a virus and a bacterium mixed in a raw material and a final product is carried out to ensure safety of a pharmaceutical product derived from human serum. When a small amount of a virus is detected by an antigen-antibody reaction or a nucleic acid amplification test, sensitivity and accuracy can be improved by preliminarily removing a nucleic acid derived from a host and a living body other than the target virus.

For example, Non-patent document 1 discloses the example to produce a vaccine derived from a Vero cell by degrading a nucleic acid with using a nucleolytic enzyme; however, a nucleolytic enzyme is expensive and a step to remove a nucleolytic enzyme and a fragmented nucleic acid is necessary.

Patent document 1 discloses that the zeolite having barium sulfate on the surface thereof can adsorb a virus from a cell culture fluid. But Patent document 1 does not disclose that a misplaced impurity derived from a host cell, such as a protein, a nucleic acid and a lipid, is adsorbed to be removed. In addition, an amount of a poorly water-soluble inorganic compound adsorbed on a carrier surface is limited.

Patent document 2 discloses that a complex depth filter medium containing silica and alumina as a filter aid can reduce all of an organic carbon (TOC) in a cell culture fluid but can recover a monoclonal antibody (mAb) with high efficiency. On the one hand, Patent document 2 discloses the data to demonstrate that turbidity and a DNA concentration cannot be reduced.

Patent document 3 discloses that turbidity of a supernatant can be reduced while an antibody concentration can be maintained by adding a cation solution and an anion solution to a cell culture medium to generate a water-insoluble solid and separating the generated solid with centrifugation, but a condition of a latter purification stage process is limited due to an increased liquid amount and an increase of ionic strength in the liquid.

Non-patent document 2 discloses the usability of removal of DNA and histone. But the accurate pH adjustment is necessary to ensure compatibility between a yield and removal of an impurity.

Patent document 4 discloses that a depth filter layer containing hydrotalcite binds to DNA but the binding ability thereof to IgG is relatively low. It is however found by the present inventor's experiment that hydrotalcite is not suitable for large-scale purification with a yield of 80% or more, since hydrotalcite also has adsorption capacity to an antibody to some extent.

### PRIOR ART DOCUMENT

### Patent Document

Patent document 1: JP 2009-42074 A
Patent document 2: JP 2016-530993 A
Patent document 3: JP 2009-508486 A
Patent document 4: JP 2011-530405 A

### Non-Patent Document

Non-patent document 1: Si-Ming Li et al., Biologicals, 42 (2014), 271-276
Non-patent document 2: Pete Gagnon et al, Journal of Chromatography A, 1374 (2014), 145-155

### DISCLOSURE OF THE INVENTION

### Problems to be Solved by the Invention

Various technologies to roughly purify a target substance by adsorbing an impurity on a solid have been developed as described above.

But, for example, the invention described in Patent document 3 is not suitable for large-scale purification of an antibody, since formation of a water-insoluble solid takes time. Also, the invention may have a problem of blockage of a pipe, since a fine crystal of a water-insoluble solid may grow in the latter stage or a water-insoluble crystal is newly generated after being separated from a cell. Furthermore, high ion strength may inhibit various analyses. If each ion concentration is reduced to solve the problems, a water-insoluble solid may not be produced in a sufficient amount and thus an impurity may not be effectively removed.

Accordingly, the objective of the present invention to provide a method for effectively and easily reducing an amount of a specific impurity in a liquid, and an adsorbing filter useful for purifying a useful substance, such as an antibody and an antibody-like molecule, used as an active ingredient of a bio-pharmaceutical product due to excellent adsorption ability to a nucleic acid and low adsorption ability to an antibody, an antibody-like molecule or the like.

### Means for Solving the Problems

The inventors of the present invention made extensive studies to solve the above-described problem. As a result, the inventors completed the present invention by finding that amounts of a nucleic acid, a nucleic acid-binding protein and a lipopolysaccharide can be reduced and thus a subsequent load for purification of a target useful substance can be reduced by contacting a target substance to be purified containing an impurity such as a nucleic acid with a water-insoluble magnesium compound.

Hereinafter, the present invention is described.

[1] A method for reducing an amount of a nucleic acid in a liquid, the method comprising the steps of:

contacting the liquid with a water-insoluble magnesium compound to adsorb at least a part of the nucleic acid on the water-insoluble magnesium compound, and then

separating the liquid from the water-insoluble magnesium compound.

[2] The method according to the above [1], wherein the water-insoluble magnesium compound is one or more selected from magnesium carbonate, magnesium hydroxide, magnesium oxide, magnesium silicate and magnesium phosphate.

[3] The method according to the above [1] or [2], wherein the liquid further comprises a useful substance.

[4] The method according to any one of the above [1] to [3], wherein the liquid is separated from the water-insoluble magnesium compound by filtration.

[5] The method according to the above [3], wherein the useful substance is one or more useful substances selected from a useful protein, a virus and a virus-like particle.

[6] The method according to the above [5], wherein the useful protein is one or more useful proteins selected from an antibody, an antibody-like molecule, a hormone, an enzyme, a growth factor, a blood protein and an antibody-binding protein.

[7] The method according to any one of the above [1] to [6], wherein the liquid is a cell culture fluid or a body fluid.

[8] An adsorbing filter comprising a layer comprising a water-insoluble magnesium compound.

[9] The adsorbing filter according to the above [8], comprising the layer consisting of a water-insoluble magnesium compound particle.

[10] The adsorbing filter according to the above [8], comprising the layer comprising the water-insoluble magnesium compound and a water-insoluble medium.

[11] The adsorbing filter according to the above [10], wherein a material of the water-insoluble medium is one or more selected from a polysaccharide, a synthetic polymer and an inorganic substance.

[12] The adsorbing filter according to the above [11], wherein the polysaccharide is one or more selected from cellulose, cellulose acetate, nitrocellulose, agarose and chitosan.

[13] The adsorbing filter according to the above [11], wherein the synthetic polymer is one or more selected from polyacrylonitrile, polyester, polyether sulfone, polypropylene and polytetrafluoroethylene.

[14] The adsorbing filter according to the above [11], wherein the inorganic substance is one or more selected from glass, silica, alumina, zirconia and barium titanate.

[15] The adsorbing filter according to any one of the above [8] to [14], wherein the water-insoluble magnesium compound is one or more selected from magnesium carbonate, magnesium hydroxide, magnesium oxide and magnesium phosphate.

### Effect of the Invention

An amount of an impurity derived from a cell, such as a nucleic acid, can be reduced and thus a target useful substance can be roughly purified by the present invention method, since the water-insoluble magnesium compound can adsorb at least a nucleic acid but does not adsorb or hardly adsorb at least an antibody and an antibody-binding protein. In addition, the present invention can be easily carried out with a low cost, since the water-insoluble magnesium compound is inexpensive and can adsorb the above-described cell-derived impurity by contacting the liquid containing the cell-derived impurity with the water-insoluble magnesium compound. Furthermore, filtration can be carried out in a short time, since the water-insoluble magnesium compound can improve filtration efficiency as a filter aid.

In addition, an impurity such as DNA can be adsorbed to be removed and a filtrate which contains a useful substance such as an antibody and an antibody-like molecule and in which an impurity concentration is reduced can be obtained by easy operation only by passing the liquid containing an impurity in addition to a useful substance such as an antibody and an antibody-like molecule through the adsorbing filter of the present invention. Also, a host cell and a fragment thereof can be concurrently physically removed. Thus, since a water-soluble impurity such as DNA can be removed and a suspended substance such as a cell can be further removed, a load of purification by subsequent chromatography can be reduced.

Accordingly, the present invention is industrially very useful, since the present invention contributes to the mass production of a useful substance such as antibody, of which demand is expected to be increased more and more in the future.

### BRIEF DESCRIPTION OF THE DRAWINGS

FIG. 1 is a graph to demonstrate a filtrate yield in the case where the water-insoluble magnesium compound was added to a CHO cell suspension and the mixture was filtrated.

FIG. 2 is a graph to demonstrate a filtrate yield in the case where the water-insoluble magnesium compound was added to a HEK cell suspension and the mixture was filtrated.

FIG. 3 is a schematic diagram of a layered device filled with basic magnesium carbonate according to the present invention.

FIG. 4 is a schematic diagram of a distributed device filled with basic magnesium carbonate according to the present invention.

### MODE FOR CARRYING OUT THE INVENTION

The present invention relates to a method for reducing an amount of a nucleic acid in a liquid by contacting the liquid comprising the nucleic acid with a water-insoluble magnesium compound to adsorb at least a part of the nucleic acid on the water-insoluble magnesium compound. Each step of the present invention method is hereinafter described, and the present invention is not restricted to the following specific examples.

1. Step to Prepare Impurity-Containing Liquid

A liquid containing a nucleic acid to be reduced is prepared in this step. This step may be carried out or may not be carried out, and it is not necessary to carry out this step in the case where the liquid is already obtained. The liquid may be an aqueous solution or a suspension. The aqueous solution means a solution in which all of the components are dissolved in water as a solvent and which does not substantively contain an insoluble component. The suspension means a liquid that may contain a solute and that contains an insoluble component. An example of the insoluble component includes a cell, a cell fragment, a cell lysate and an aggregate of a component derived from a cell, such as an aggregated protein. The insoluble component may be dispersed or may be precipitated in a solution.

A nucleic acid is a biological polymer formed by binding nucleotides consisting of a base, a sugar and a phosphate through a phosphodiester bond and exists as RNA or DNA in a living body. A nucleic acid as DNA or RNA may be an impurity regardless of the composition or the length thereof. In addition, a fragmented nucleic acid and a conjugate with a protein, such as chromosome, may be an impurity. A plasmid used for transient expression in addition to the nucleic acid derived from a cell may be also impurity in the case where the liquid containing a useful substance is a culture fluid. When the liquid containing a useful substance is a body fluid, cell free DNA and cell free RNA may be also an impurity in addition to the nucleic acid derived from a cell. When the liquid containing a useful substance contains a virus, DNA and RNA leaked from the virus may be also an impurity.

A genomic nucleic acid has problems of increase of viscosity of the liquid containing a useful substance and accumulation and residue in the purified product and additionally causes deterioration of sensitivity and accuracy of analysis, since a genomic nucleic acid has a long chain structure and often forms a complex with a protein. It is therefore important to remove a genomic nucleic acid. It is important for a cell culture fluid to remove a long chain nucleic acid and a fragmented DNA leaked by cell breakage.

An amount of the other impurity derived from a cell in addition to a nucleic acid may be reduced by the present invention method. An example of such an impurity includes a nucleic acid-binding protein and a lipopolysaccharide. A nucleic acid-binding protein is mainly a DNA-binding protein such as histone. The four kinds of core histone among proteins called as histone forms a histone octamer consisting of two molecules each, forms a nucleosome with DNA, and a linker histone binds to the DNA between the nucleosomes. Thus, the molecular weight of a DNA-binding protein contained in a cell breakage liquid and a cell lysate may be relatively large. A lipopolysaccharide is a complex of a lipid and polysaccharide bound through a covalent bind and a main body of an endotoxin mainly existing as an outer membrane component of gram-negative bacteria.

The above-described liquid may contain a useful substance to be purified. An example of the useful substance includes a useful protein such as an antibody, an antibody-like molecule, an antibody-binding protein, an enzyme, a growth factor, a hormone, a cytokine and a plasma protein; and a virus and a virus-like particle used for gene therapy, and research, development and production of a vaccine.

The “antibody or antibody-like molecule” that may be a target to be purified by the present invention may be an antibody or an antibody-like molecule that are industrially useful, is a functional protein having a polypeptide structure, and may have a secondary structure such as alpha helix and beta sheet structure in the molecule, have a sugar chain, be modified by a sugar, phosphorylation and tyrosination, and be coordinated by a metal. In addition, the antibody or antibody-like molecule may be a natural protein and peptide, and may be produced by a genetic recombination technology. The function thereof may be improved. The antibody or antibody-like molecule may have a structure consisting of a functional part, and may be prepared by connecting different functional parts or the same functional parts. The antibody or antibody-like molecule may be crosslinked by a disulfide bond of cysteine residues in the molecule, may be crosslinked by a disulfide bond of cysteine residues between the molecules, and may contain a subunit structure through a non-covalent bond. The antibody or antibody-like molecule may be a protein connected by chemical modification, and a function may be added thereto by chemical modification of a protein and addition of a functional molecule.

The antibody or antibody-like molecule is not particularly restricted and is exemplified by polyclonal antibody, monoclonal antibody, human antibody, humanized antibody, chimeric antibody, single chain antibody, heavy chain antibody, polyvalent antibody, Fab, F(ab′), F(ab′)₂, Fc, Fc-fusion protein, bispecific antibody, heavy chain (H chain), light chain (L chain), single chain Fv (scFv), sc(Fv)₂, disulfide-linked Fv (sdFv), diabody and antibody-like molecule target peptide (micro antibody). The antibody or the antibody-like molecule may be any one of an Fc-containing protein, such as immunoglobulin and Fc-fusion protein having an Fc part, and a low-molecular antibody, such as the above-described Fab, F(ab′), F(ab′)₂, Fc, heavy chain (H chain), light chain (L chain), single chain Fv (scFv), sc(Fv)₂, disulfide-linked Fv (sdFv), single chain antibody, heavy chain antibody, multivalent antibody, bispecific antibody, diabody and antibody-like molecule target peptide (micro antibody) in the present invention.

An example of the enzyme includes lipase, protease, steroid synthetase, kinase, phosphatase, xylanase, esterase, methylase, demethylase, oxidase, reductase, cellulase, aromatase, collagenase, transglutaminase, glycosidase and chitinase.

An example of the growth factor includes epidermal growth factor (EGF), insulin-like growth factor (IGF), transforming growth factor (TGF), nerve growth factor (NGF), brain-derived neurotrophic factor (BDNF), vascular endothelial growth factor (VEGF), granulocyte colony-stimulating factor (G-CSF), granulocyte-macrophage colony-stimulating factor (GM-CSF), platelet-derived growth factor (PDGF), erythropoietin (EPO), thrombopoietin (TPO), fibroblast growth factor (FGF) and hepatocellular growth factor (HGF).

An example of the hormone includes insulin, glucagon, somatostatin, growth hormone, parathyroid hormone, prolactin, leptin and calcitonin. An example of the cytokine includes interleukin, interferon (IFN α, IFN β and IFN γ) and tumor necrosis factor (TNF).

An example of the plasma protein includes thrombin, serum albumin, VII factor, VIII factor, IX factor, X factor and tissue plasminogen activator.

The antibody binding protein is not particularly restricted as long as the antibody binding protein is a protein having a specific binding ability to an antibody and is exemplified by Protein A, Protein G, Protein L, Fcγ receptor, antibody binding domains thereof and variants thereof of which binding ability to an antibody or an antibody-like molecule is maintained or improved.

The virus is not particularly restricted as long as the virus itself or a part thereof should be purified. An example of a non-enveloped virus includes adeno-associated virus, adenovirus, enterovirus, parvovirus, papovavirus, human papillomavirus, rotavirus, coxsackievirus, sapovirus, norovirus, poliovirus, echovirus, coronavirus, hepatitis A virus, hepatitis E virus, rhinovirus and astrovirus. An adeno-associated virus has an AAV capsid serotype selected from the group consisting of AAV-1, AAV-2, AAV-3, AAV-4, AAV-5, AAV-6, AAV-7, AAV-8, AAV-9, AAV-10, AAV-11, AAV-12, AAV-13, AAV-14, AAV-15 and AAV-16. An example of an enveloped virus includes retrovirus, lentivirus, hemagglutinating virus of Japan, herpes simplex virus, vaccinia virus, measles virus, baculovirus and influenza virus.

The virus-like particle means all of or a part of a virus outer shell protein that mainly constitutes a capsid. The virus-like particle does not raise infection concerns, since the virus-like particle does not contain a nucleic acid. But the virus-like particle can be used as an active ingredient of a vaccine, since the virus-like particle causes an immune reaction.

The liquid to be treated with the specific water-insoluble magnesium compound in the present invention is not particularly restricted as long as the liquid contains the above-described impurity, and may contain a useful substance. An example of the liquid includes culture fluid, culture supernatant, cultivated cell, aqueous suspension of cell fragment and cell lysate and extract thereof, living body extract, liquid containing a virus or a virus-like particle, and body fluid. The aqueous solution and the aqueous suspension may contain an organic solvent. The cell may be a virus-infected cell. The liquid containing a virus or a virus-like particle can be produced by infecting a cultivated cell or a fertilized egg of a chicken with the virus and growing the virus and the virus-like particle in a culture fluid or a body fluid. The body fluid means a liquid with which the space between tissues, body cavity, a tube spreading systemically and a circulatory system are filled in an animal body, or a liquid secreted or excreted inside or out of the body. For example, the liquid to be treated may be a chorioallantoic fluid obtained by inoculating blood or the virus strain into the inside of the allantoic cavity of a hen egg to be cultivated and being separated.

The above-described cell to be cultivated may be a naturally derived and is preferably a recombinant host cell. The host is not particularly restricted as long as the host is used for producing a useful substance and is an animal cell, a plant cell, an insect cell or a microorganism cell to produce a useful protein by being transformed with expression vector and a gene fragment containing DNA encoding the useful protein or to be infected with a virus. The gene recombinant means a host cell transformed by introducing an expression vector or a gene fragment having a base sequence encoding an amino acid sequence of a useful protein and a promotor that is connected to the base sequence to be functioned in a host in the present invention.

The above-described liquid may contain the other impurity in addition to the above-described impurity and a useful substance. The other impurity is not particularly restricted and is exemplified by an aggregated protein, a plasmid, a culture medium component and a plasmid DNA. The solvent of the liquid may be a buffer solution.

2. Step of Treatment by Water-Insoluble Magnesium Compound

An amount of the above-described impurity is reduced by contacting the liquid containing the above-described impurity with the water-insoluble magnesium compound in order to adsorb at least a part of the impurity on the water-insoluble magnesium compound in this step. As a result, when the liquid contains a useful substance, the useful substance is roughly purified by reducing an amount of the impurity. The term “purification” means that a ratio of the impurity to a useful substance in the liquid before being contacted with the water-insoluble magnesium compound is reduced.

The term “water-insolubility” means a degree to dissolve the magnesium compound within 30 minutes in the case where the magnesium compound powder is added to purified water and the mixture is vigorously shaken at 20±5° C. for 30 seconds every 5 minutes in this disclosure, and specifically means an amount of purified water required for dissolving 1 g of the magnesium compound is 100 mL or more. The amount of the purified water is preferably 1000 mL or more.

The water-insoluble magnesium compound is not particularly restricted as long as the water-insoluble magnesium compound is insoluble in water and does not have an adsorption ability to a target useful substance such as an antibody or has the low adsorption ability but adsorbs an impurity such as a nucleic acid derived from a host cell. An example of the water-insoluble magnesium compound includes one or more selected from magnesium carbonate, magnesium hydroxide, magnesium oxide and magnesium phosphate. For example, the water-insoluble magnesium compound may be basic magnesium carbonate: mMgCO₃.Mg(OH)₂.nH₂O (wherein m is 3 or more and 5 or less, and n is 3 or more and 7 or less). Basic magnesium carbonate is obtained as precipitation by adding an alkali metal carbonate such as sodium carbonate and potassium carbonate to an aqueous solution of a magnesium salt. The water-soluble magnesium-containing compound is preferably basic magnesium carbonate and/or magnesium oxide. On the one hand, it is experimentally found by the present inventors that a water-insoluble magnesium compound that contains a metal ion other than magnesium, such as hydrotalcite: Mg₆Al₂CO₃(OH)₁₆.4H₂O, adsorbs an antibody or the like to a certain degree, and is therefore preferably not used as the water-insoluble magnesium compound in the present invention.

The size of the water-insoluble magnesium compound may be adequately adjusted, and for example, the average particle diameter thereof may be adjusted to 0.1 μm or more and 1000 μm or less. When the average particle diameter is 1000 μm or less, the water-insoluble magnesium compound can more efficiently adsorb an impurity due to a sufficiently large specific surface area. When the average particle diameter is 0.1 μm or more, excessive energy for pulverization is not needed. The average particle diameter is preferably 1 μm or more and more preferably 10 μm or more in terms of handleability in the case where a column is filled with the water-insoluble magnesium compound. The average particle diameter is measured by a laser diffraction particle size measuring device in this disclosure, and an average particle diameter is based on a volume, a weight, a number or the like and the average particle diameter is preferably based on a volume.

A usage amount of the water-insoluble magnesium compound may be adjusted depending on an amount of an impurity in the liquid, and for example, 0.01 g or more and 100 g or less of the water-insoluble magnesium compound to 100 mL of the above-described liquid may be used. The ratio is preferably 15 g/100 mL or less. In addition, 0.01 w/v % or more and 100 w/v % or less of the water-insoluble magnesium compound to the above-described liquid may be used, and the ratio is preferably 1 w/v % or more and 15 w/v % or less.

A condition to contact the above-described liquid with the water-insoluble magnesium compound may be appropriately selected. For example, the water-insoluble magnesium compound is added to the liquid, and the mixture may be shaken or stirred. The temperature at the time may be an atmospheric temperature, and may be specifically adjusted to 0° C. or higher and 40° C. or lower. The temperature is preferably 1° C. or higher, more preferably 10° C. or higher or 15° C. or higher, and preferably 30° C. or lower, more preferably 25° C. or lower. The time for the contact may be adjusted to 1 second or more and 10 hours or less.

3. Separation Step

The water-insoluble magnesium compound on which at least a part of a nucleic acid contained in the liquid is separated from the liquid in this step. The separation means is not particularly restricted as long as the water-insoluble magnesium compound and the liquid can be separated by the means, and is exemplified by centrifugation and filtration.

After the liquid and the water-insoluble magnesium compound are separated, a useful substance is mainly dispersed in the liquid phase and all of or a part of an impurity is adsorbed on the water-insoluble magnesium compound. Although a part of a useful substance may be adsorbed on the water-insoluble magnesium compound and a part of an impurity is dissolved in the liquid phase in some cases, at least a total amount of the impurity in the liquid can be reduced and a useful substance is concentrated in the liquid phase.

The impurity may be adsorbed on the water-insoluble magnesium compound by filling a column with the water-insoluble magnesium compound and passing the above-described liquid through the column. Adsorption of an impurity and separation of the liquid phase from the water-insoluble magnesium compound can be concurrently carried out in such a case. An amount of the water-insoluble magnesium compound to fill a column and a flow velocity of the above-described liquid are preferably adjusted to the extent that at least the above-described impurity is sufficiently adsorbed on the water-insoluble magnesium compound.

The water-insoluble magnesium compound is also useful as a filter aid to improve filterability. The term “filterability” includes the concept that filtration is successfully carried out with suppressing clogging of a filter. For example, clogging of a filter can be suppressed in the case where the liquid as a suspension is contacted with the water-insoluble magnesium compound and then an insoluble component containing the water-insoluble magnesium compound is separated by filtration. Thus, the water-insoluble magnesium compound is preferably separated from the liquid by filtration.

At least a part of the above-described impurity and a relatively large insoluble component such as a cell are separated to be removed by the above-described Step 2 and Step 3. When the liquid contains a useful substance, the useful substance is preferably further purified by chromatography. An amount of the above-described impurity or the other impurity may be further reduced by a general treatment step before chromatography. Such a general treatment step is hereinafter described.

4. Treatment Step by Activated Carbon

The liquid containing an impurity is contacted with activated carbon in this step. This step may be carried out before the above-described Step 2, after the Step 2, or simultaneously with the Step 2 by using the water-insoluble magnesium compound and activated carbon in combination. This step may be carried out or may not be carried out.

Activated carbon means a porous substance produced by burning charcoal, palm shell or the like to develop pores and is excellent in adsorption performance. A general specific surface area of activated carbon is about 800 m²/g or more and about 2500 m²/g or less.

An average pore diameter of activated carbon is not particularly restricted and is generally 0.1 nm or more and 20 nm or less, preferably 0.5 nm or more and 5.0 nm or less, more preferably 2.0 nm or more and 5.0 nm or less, and even more preferably 3.0 nm or more and 5.0 nm or less. An average pore diameter of activated carbon can be calculated by BJH method on the basis of a nitrogen absorption isothermal curve.

A purification means using activated carbon is not particularly restricted in the present invention and is exemplified by a batch method, a membrane treatment method and a column chromatography method. A preferred activated carbon form may be selected depending on each means. Activated carbon can be also used as needed in a particle form prepared by enclosing activated carbon in a porous polymer or a gel, a membrane prepared by using a supporting agent or a fiber such as polypropylene and cellulose to adsorb, immobilize or shape activated carbon, or a cartridge form.

A usage amount of activated carbon may be adjusted depending on a concentration of an impurity in the liquid to be treated, and for example, 0.5 g or more and 5 g or less of activated carbon may be used to 100 mL of the liquid.

Activated carbon may be added to the impurity-containing liquid and the mixture may be shaken or stirred, or a column is filled with activated carbon similarly to the water-insoluble magnesium compound with respect to a condition to contact the impurity-containing liquid and activated carbon. When this Step 4 and the above-described Step 2 are concurrently carried out, the water-insoluble magnesium compound and activated carbon may be mixed to be used. After the impurity-containing liquid and activated carbon are contacted, the liquid phase is separated from the activated carbon. The above-described Step 3 may be carried out after this Step 4 and the above-described Step 2 are concurrently carried out.

5. Treatment Step by Flocculant

The liquid containing an impurity is treated with a flocculant in this step. This step may be carried out before the Treatment step 2 by the water-insoluble magnesium compound and/or the Treatment step 4 by activated carbon, after the Treatment step 2 and/or the Treatment step 4, or concurrently with the Treatment step 2 and/or the Treatment step 4 by using the water-insoluble magnesium compound and/or activated carbon and a flocculant in combination. This step may be carried out or may not be carried out.

An example of the flocculant includes caprylic acid, polyamine, divalent cation, polyetherimine, chitosan, polyethylene glycol, polyvinyl alcohol, polyvinylpyrrolidone and poly(diallyldimethylammonium chloride) (pDADMAC). An example of the divalent cation includes Ca²⁺, Mg²⁺, Cu²⁺, Co²⁺, Mn²⁺, Ni²⁺, Be²⁺, Sr²⁺, Ba²⁺, Ra²⁺, Zn²⁺, Cd²⁺, Ag²⁺, Pd²⁺ and Rh²⁺. The divalent cation can be used in a free state or as a hydrochloride salt, a sulfate salt or a citrate salt.

A usage amount of the flocculant may be adjusted depending on a concentration of an impurity in the liquid to be treated. For example, when the flocculant is polyamine or polyetherimine, 0.01 w/v % or more and 10 w/v % or less of the flocculant may be used and 0.1 w/v % or more and 1 w/v % or less of the flocculant may be more preferably used. When the flocculant is caprylic acid, chitosan, polyethylene glycol, polyvinyl alcohol or polyvinylpyrrolidone, 0.01 w/v % or more and 10 w/v % or less of the flocculant may be used and 1 w/v % or more and 5 w/v % or less of the flocculant may be more preferably used. When the flocculant is pDADMAC, 0.01 w/v % or more and 0.1 w/v % or less of the flocculant may be used and 0.1 w/v % or more and 0.5 w/v % or less of the flocculant may be more preferably used. When the flocculant is a divalent cation, a divalent cation may be added in an amount of 1 mM or more and 100 mM or less and more preferably added in an amount of 2 mM or more and 50 mM or less.

The flocculant may be added to the impurity-containing liquid and the mixture may be shaken or stirred or a column may be filled with the flocculant similarly to the water-insoluble magnesium compound with respect to the condition to contact the impurity-containing liquid and the flocculant. When this Step 5 and the Treatment step 2 by the water-insoluble magnesium compound and/or the Treatment step 4 by activated carbon are concurrently carried out, the flocculant and the water-insoluble magnesium compound and/or activated carbon may be mixed to be used. After the impurity-containing liquid and the flocculant are contacted, the liquid phase is separated from the flocculant. The above-described Step 3 may be carried out after this Step 5 and the above-described Step 2 and the above-described Step 4 are concurrently carried out.

6. Treatment Step by Endonuclease

The liquid containing an impurity is treated with endonuclease in this step. This step may be carried out before each of the above-described step, after each of the above-described step, or concurrently with each of the above-described step by using one or more selected from the water-insoluble magnesium compound, activated carbon and the flocculant with endonuclease in combination. This step may be carried out or may not be carried out.

Endonuclease is one of DNase and has a function to cleave a base sequence even at a central part. An example of endonuclease includes Benzonase manufactured by Millipore and KANEKA Endonuclease manufactured by KANEKA as commercialized products.

A usage amount of endonuclease may be adjusted depending on a concentration of DNA in the liquid and for example, is preferably 10 U/mL or more and more preferably 100 U/mL or more in the liquid. The upper limit is not particularly restricted and the usage amount is preferably 10,000 U/mL or less.

7. Step of Other Purification

The above-described step, especially the above-described Treatment step 2, may be preferably carried out in any stages, for example, before or after a general step to reduce an impurity, such as a membrane treatment and a column treatment described below. When the above-described step is carried out before a membrane treatment and a column treatment, decrease of adsorption capacity of a chromatography carrier, decrease of separation ability, decrease of processing speed due to increase of back pressure, and decrease of a lifetime of a carrier due to washing or decrease of regeneration efficiency can be expected to be suppressed. Decrease of treatment capacity per a unit membrane area, increase of back pressure, decrease of processing speed, and decrease of a lifetime of a carrier due to washing or decrease of regeneration efficiency can be also expected to be suppressed in a membrane treatment process. Accordingly, one of preferred embodiments of the present invention to further subject the liquid which contains a useful substance and in which an impurity is reduced by the above-described step to a column treatment and a membrane filtration treatment is preferred. In other words, the present invention method can be used before a column treatment and a membrane filtration treatment.

The liquid containing a useful substance can be subjected to purification by a column treatment such as chromatography. Such chromatography is not particularly restricted as long as a useful substance as a target can be recovered and purified, and is exemplified by anion exchange chromatography, cation exchange chromatography, hydrophobic chromatography, hydroxyapatite chromatography, mixed mode chromatography and affinity chromatography. One chromatographic method may be used alone, and two or more chromatographic methods may be used in combination. The above-described step, especially the above-described Treatment step 2, may be preferably carried out in any stages, for example, before or after the above-described chromatography step.

After a useful substance is purified, the useful substance may be concentrated by reducing a solvent amount and a solvent may be exchanged.

An amount of an impurity can be determined by a commercially available assay kit in addition to absorption spectrometry, electrophoresis and HPLC in any stages. For example, an amount of a nucleic acid can be determined by absorption spectrometry of wavelength of maximum absorption of a nucleic acid, liquid chromatography isotope-dilution mass spectrometry (LC IDMS), fluorescence analysis using a fluorescent reagent, emission spectrometry, mass spectrometry, chromatography analysis, a combination thereof, gel electrophoresis, q-PCR and a next-generation DNA sequencer. An example of an emission spectrometry includes inductive coupling plasma emission analysis method (ICP OES) and inductively coupled plasma mass spectrometry (ICP MS). An amount of a protein derived from a CHO cell can be determined by using CHO HCP ELISA kit manufactured by Cygnus. If an assay kit for a target impurity protein is not commercially available, a system to detect the target impurity protein can be prepared by immunizing an animal such as a chicken with the impurity protein. A load of chromatography can be reduced and thus more efficient purification becomes possible by removing a part of an impurity other than a useful substance before chromatography.

The above-described method for reducing a nucleic acid can be carried out by, for example, the adsorbing filter of the present invention. The adsorbing filter of the present invention comprises a layer comprising the water-insoluble magnesium compound. The term “adsorbing filter” means a filter that can reduce an amount of an impurity such as DNA by adsorbing at least a part of the impurity and that can physically prevent the transmission of a larger substance than pores. The description of the water-insoluble magnesium compound or the like in the description of the method for reducing a nucleic acid according to the present invention is applied to the water-insoluble magnesium compound or the like contained in the adsorbing filter of the present invention.

The layer that constitutes the adsorbing filter of the present invention and contains the water-insoluble magnesium compound is not particularly restricted as long as the layer has necessary thickness and a pore through which the liquid containing a useful substance can pass, and may or may not contain a component other than the water-insoluble magnesium compound. The layer is hereinafter referred to as “water-insoluble magnesium compound-containing layer”. The water-insoluble magnesium compound may be a particle such as a spherical particle processed to be porous and a blockish fragment. The filterability of the adsorbing filter according to the present invention is improved by the water-insoluble magnesium compound-containing layer.

For example, the water-insoluble magnesium compound-containing layer may consist of the water-insoluble magnesium compound. The layer consisting of the water-insoluble magnesium compound means a layer which substantively consists of the water-insoluble magnesium compound particle and to which a component other than the water-insoluble magnesium compound is not intentionally added even though an inevitable impurity and an unavoidable mixture are acceptable.

The layer may contain a component other than the water-insoluble magnesium compound as long as the layer contains the water-insoluble magnesium compound as a main component. The other component is not particularly restricted as long as the other component is a water-insoluble medium that is insoluble in water and has low adsorption ability to a target useful substance. The other component is exemplified a water-insoluble medium consisting of one or more selected from activated carbon, a polysaccharide, a synthetic polymer and an inorganic substance, and is preferably a water-insoluble medium consisting of one or more selected from a polysaccharide, a synthetic polymer and an inorganic substance from the point that non-specific adsorption is particularly small. The filterability, liquid permeability and/or impurity removability of the water-insoluble magnesium compound-containing layer may be improved by mixing a water-insoluble medium in some cases. It is preferred that the water-insoluble magnesium compound and the water-insoluble medium are respectively homogeneously dispersed in the water-insoluble magnesium compound-containing layer. The shape of the water-insoluble medium is not particularly restricted and may be, for example, a particle and a fiber. The condition of the water-insoluble magnesium compound and the water-insoluble medium is not particularly restricted as long as at least a part of the water-insoluble magnesium compound is exposed on the surface to exert adsorption ability, and may be a mixed condition, adhered condition or bound condition.

An example of a polysaccharide includes a cellulose such as cellulose, cellulose acetate and nitrocellulose; agarose; and chitosan. An example of a synthetic polymer includes polyacrylonitrile, polyester, polyether sulfone, polypropylene and polytetrafluoroethylene. An example of an inorganic substance includes diatomite, pearlite, glass, silica, alumina, zirconia and barium titanate.

When the water-insoluble magnesium compound-containing layer contains a water-insoluble medium, a usage amount of the water-insoluble medium may be adequately adjusted depending on the form and the amount of the insoluble substance in the liquid to be treated, the viscosity of the liquid to be treated or the like, and for example, a ratio of the water-insoluble medium to the total of the water-insoluble magnesium compound and the water-insoluble medium may be adjusted to 1 mass % or more and 99 mass % or less. When the ratio is 1 mass % or more, the effect to improve the filterability and the liquid permeability can be obtained more surely. When the ratio is 99 mass % or less, the water-insoluble magnesium compound may be more surely exposed on the surface and the effective amount of the water-insoluble magnesium compound as the main component can be ensured more surely. The ratio is preferably 2 mass % or more, more preferably 5 mass % or more, and preferably 50 mass % or less, more preferably 30 mass % or less.

Since the liquid can pass through the layer in one direction, for example, from the upper side to the lower side, the water-insoluble magnesium-containing compound or the mixture of the water-insoluble medium and the water-insoluble magnesium-containing compound that constitute the layer is not needed to be immobilized on the surface of a support material and may be merely accumulated on a support material. A support material may be set for the water-insoluble magnesium compound-containing layer. In particular, when the layer contains the fine water-insoluble magnesium compound as a main component, the water-insoluble magnesium compound-containing layer is preferably sandwiched from the upper and the lower sides using support materials such as membranes in order to prevent the leakage of the fine particle from the adsorbing filter.

The size and the thickness of the water-insoluble magnesium compound-containing layer may be adequately adjusted depending on whether the water-insoluble medium is used or not, an amount of the water-insoluble medium and an amount of the liquid to be treated as long as the concentration of the target impurity can be reduced down to a predetermined value or less. For example, an amount of the water-insoluble magnesium compound constituting the water-insoluble magnesium compound-containing layer or a total amount of the water-insoluble magnesium compound and the water-insoluble medium may be adjusted to 0.0001 times or more by mass and 1 time or less by mass to an amount of the liquid to be treated. When the ratio is 0.0001 times or more by mass, the liquid can be successively treated more surely, since there are little possibilities of the clogging and saturation of the adsorbed impurity. When the ratio is 1 time or less by mass, the processing system may not become excessively large in comparison with the amount of the liquid to be treated. The ratio is preferably 0.0005 times or more by mass, more preferably 0.001 times or more by mass, and preferably 0.5 times or less by mass, more preferably 0.1 times or less by mass. The above-described amount means a total amount of the water-insoluble magnesium-containing compound and the like in the case of the two or more water-insoluble magnesium compound-containing layers.

The adsorbing filter of the present invention may contain the two or more water-insoluble magnesium compound-containing layers. For example, the number of the water-insoluble magnesium compound-containing layer may be 1 or more and 3 or less, is preferably 2 or less, and may be 1. The thickness of the water-insoluble magnesium compound-containing layer may be adjusted to, for example, 1 μm or more and 1000 cm or less.

The amount of the water-insoluble magnesium compound or the total amount of the water-insoluble medium and the water-insoluble magnesium compound that constitute the water-insoluble magnesium compound-containing layer is preferably adjusted so that the time to contact the layer and the liquid to be treated becomes 10 seconds or more and 60 minutes or less depending on the velocity of the liquid to be treated to be supplied to the water-insoluble magnesium compound-containing layer. When the contact time is included in the above-described range, an impurity can be efficiently removed. The contact time is preferably 20 seconds or more, more preferably 60 seconds or more, and preferably 30 minutes or less, more preferably 20 minutes or less. The contact time means a total contact time with each layer in the case of the two or more water-insoluble magnesium compound-containing layers.

The adsorbing filter may contain a layer other than the water-insoluble magnesium compound-containing layer. For example, the adsorbing filter may contain a support layer having a pore diameter to a degree that the water-insoluble magnesium compound particle can be kept just below the water-insoluble magnesium compound-containing layer. In addition, the adsorbing filter may contain a general support material layer on the water-insoluble magnesium compound-containing layer or below the support layer. The material of the support material layer is exemplified by one or more water-insoluble media selected from activated carbon, a polysaccharide, a synthetic polymer and an inorganic substance, and is preferably one or more water-insoluble media selected from a polysaccharide, a synthetic polymer and an inorganic substance from the standpoint of especially less non-specific adsorption. An example of a polysaccharide includes a cellulose such as cellulose, cellulose acetate and nitrocellulose; agarose; and chitosan. An example of a synthetic polymer includes polyacrylonitrile, polyester, polyether sulfone, polypropylene and polytetrafluoroethylene. An example of an inorganic substance includes diatomite, pearlite, glass, silica, alumina, zirconia and barium titanate.

The pore diameter of the support material layer may be adequately adjusted and may be adjusted to, for example, 0.1 μm or more and 100 μm or less to capture a cell fragment or the like in the support material layer and prevent the leakage of the water-insoluble magnesium compound. When the pore diameter of the support material layer product is described in a catalog, the catalog value may be referred to as the pore diameter. When the pore diameter is not described in a catalog, the average diameter may be directly determined using an enlarged photograph or determined as an estimate value using a Gurley air permeability tester.

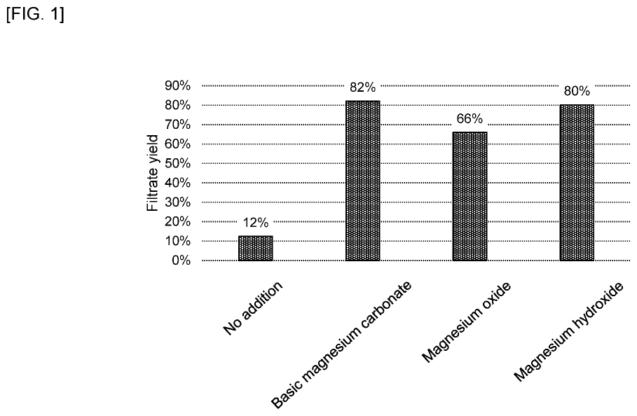

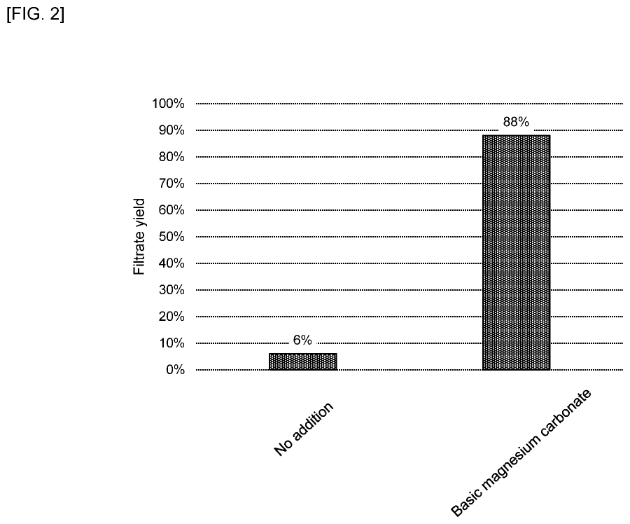

For example, a schematic diagram of a layered device filled with basic magnesium carbonate according to the present invention is shown as FIG. 1. The device of FIG. 1 is a depth filter device. A polytetrafluoroethylen (PTFE) filter 1, a depth filter 3, the water-insoluble magnesium compound-containing layer 2 consisting of the water-insoluble magnesium compound and a PTFE filter 1 are layered in order from the bottom layer in a filter holder. FIG. 2 is a schematic diagram of a distributed device filled with basic magnesium carbonate according to the present invention. A PTFE filter 1, the water-insoluble magnesium compound-containing layer 4 consisting of the mixture of the water-insoluble magnesium compound and the water-insoluble medium, and a PTFE filter 1 are layered in order from the bottom layer in a filter holder.

The adsorbing filter of the present invention may be distributed as a finished product in which the water-insoluble magnesium compound-containing layer is formed in a filter holder. Alternatively, the water-insoluble magnesium compound-containing layer may be formed for use by filling a filter folder with the water-insoluble magnesium compound.

The embodiment of the adsorbing filter according to the present invention is not particularly restricted. For example, the water-insoluble magnesium compound-containing layer may be layered on the other filter. Alternatively, the water-insoluble magnesium compound-containing layer sheet is formed from the water-insoluble magnesium compound, and then the sheet may be shaped into a pleat or a hollow fiber. In addition, a depth filter, a syringe filter and a cartridge for liquid treatment may be produced by inserting the water-insoluble magnesium compound-containing layer in a package.

The adsorbing filter of the present invention can adsorb a water-soluble impurity derived from a cell, such as DNA and histone, can be used for purifying a target useful substance such as an antibody and an antibody-like molecule, and additionally can physically separate a cell, a cell fragment and a cell lysate obtained with using a surfactant by filtration. Specifically, the above-described impurity can be removed while a useful substance is purified by passing the liquid containing the useful substance through the adsorbing filter of the present invention. The adsorbing filter can be preferably used in a former stage or a latter stage of a step to purify a general protein, such as a membrane treatment and a column treatment. When the purification method of the present invention is carried out in a former stage of a membrane treatment and a column treatment, decrease of adsorption capacity of a chromatography carrier, decrease of separation capability, decrease of treatment rate due to increase of back pressure, decrease of a lifetime of a carrier due to washing or decrease of regeneration efficiency can be suppressed. In addition, decrease of treatment capacity per a unit membrane area, increase of back pressure, decrease of processing speed, and decrease of a lifetime of a carrier due to washing or decrease of regeneration efficiency can be suppressed in a membrane treatment process. Accordingly, it is a preferred one of preferred embodiments of the present invention that the useful substance such as an antibody and an antibody-like molecule is purified by the purification method of the present invention and then is further subjected to a column treatment and a membrane filtration treatment. In other words, the adsorbing filter of the present invention can be used before a column treatment and a membrane filtration treatment. Furthermore, the adsorbing filter of the present invention can be used for a pretreatment of a sample liquid in examination and diagnosis using an equivalent of the above-described useful substance as an index.

The condition to pass a solution or a suspension containing a target useful substance through the adsorbing filter of the present invention may be appropriately determined. For example, the velocity of the solution or the suspension to pass through the adsorbing filter may be adjusted to 1 cm/hr or more and 10 m/hr or less. When the velocity is 1 cm/hr or more, the adsorbing filter may sufficiently adsorb an impurity. When the velocity is 10 m/hr or less, treatment efficiency may be sufficiently ensured. The velocity is preferably 5 cm/hr or more, more preferably 10 cm/hr or more, and preferably 5 m/hr or less, more preferably 1 m/hr or less.

The temperature in the case where the solution or the suspension containing a target useful substance is passed through the adsorbing filter of the present invention is may be an atmospheric temperature and may be specifically adjusted to 0° C. or higher and 40° C. or lower. The temperature is preferably 1° C. or higher, more preferably 10° C. or higher or 15° C. or higher, and preferably 30° C. or lower, more preferably 25° C. or lower.

After the liquid passes through the adsorbing filter of the present invention, not only a cell or the like is removed from the liquid but also a concentration of an impurity such as DNA and a DNA-binding protein is reduced. The liquid may be treated with a conventional adsorbent such as activated carbon to further reduce an impurity concentration. The liquid may be further subjected to affinity chromatography, ion-exchange chromatography, gel filtration chromatography or the like to purify a target useful substance. A load on chromatography can be reduced and thus more efficient purification becomes possible by removing a part of an impurity before chromatography. In addition, when an impurity can be reduced by a filtration step that does not impair a yield between steps, the reduction of an impurity is not needed to be prioritized at the expense of the yield of a chromatography step and thus the total purification yield through a whole production step can be improved. Furthermore, since an impurity is a residual contaminant of a material for purification such as chromatography and thus causes decrease of a lifetime and decrease in performance of the material, introduction of a simple impurity removal process is expected to contribute reduction of material cost for production due to extension of life and maintenance of performance of the material for purification.

The present application claims the benefit of the priority dates of Japanese patent applications No. 2020-33132 and No. 2020-33216 filed on Feb. 28, 2020. All of the contents of the Japanese patent applications No. 2020-33132 and No. 2020-33216 filed on Feb. 28, 2020, are incorporated by reference herein.

### EXAMPLES

Hereinafter, the present invention is described in more detail with Examples. The present invention is however not restricted to the following Examples in any way, and it is possible to work the present invention according to the Examples with an additional appropriate change within the range of the above descriptions and the following descriptions. Such a changed embodiment is also included in the technical scope of the present invention. Commercially available reagents were used in the following Examples unless otherwise stated.

### Example 1: Adsorption and Removal of DNA by Using Basic Magnesium Carbonate

Basic magnesium carbonate was added to a 1 g/L salmon sperm DNA solution in a concentration of 1 w/v % or 10 w/v %, and the mixture was stirred using a mix rotor for 2 hours. Then, the mixture was centrifuged at 15,000 rpm for 5 minutes to obtain the supernatant as the treated liquid. The DNA concentrations in the DNA solution before the treatment and in the treated liquid were determined on the basis of the absorbance of UV 260 nm. The similar experiment was conducted except that basic magnesium carbonate was not added for comparison. The result is shown in Table 1.

TABLE 1

| Additive | |
|---|---|
| amount | |
| of basic | |
| magnesium | DNA |
| carbonate | concentration |
| 0 w/v % | 1.00 mg/mL |
| 1 w/v % | 0.76 mg/mL |
| 10 w/v % | 0.03 mg/mL |

It was experimentally demonstrated as the result shown in Table 1 that basic magnesium carbonate can adsorb DNA to be removed.

### Example 2: Adsorption and Removal of Endotoxin by Using Basic Magnesium Carbonate

Basic magnesium carbonate was added to a 0.4 EU/mL endotoxin solution in a concentration of 1 w/v %, and the mixture was stirred using a mix rotor for 2 hours. Then, the mixture was centrifuged at 15,000 rpm for 5 minutes to obtain the supernatant as the treated liquid. The endotoxin concentrations in the endotoxin solution before the treatment and in the treated liquid were determined using Portable Endotoxin Testing System (“Endosafe® Nexgen PTS” manufactured by Charles River). The similar experiment was conducted except that basic magnesium carbonate was not added for comparison. The result is shown in Table 2.

TABLE 2

| Additive | | |
|---|---|---|
| amount | | |
| of basic | | |
| magnesium | Endotoxin | |
| carbonate | concentration | |
| 0 w/v % | 0.425 | EU/mL |
| 1 w/v % | <0.05 | EU/mL |

It was found from the result shown in Table 2 that basic magnesium carbonate can also adsorb endotoxin to be removed.

### Example 3: Adsorption and Removal of DNA, HCP and Histone from Culture Supernatant of Animal Cell by Using Basic Magnesium Carbonate

Basic magnesium carbonate was added to a culture supernatant of an animal cell containing monoclonal antibody (IgG) in a concentration of 1 w/v %, and the mixture was stirred at room temperature using a mix rotor for 18 hours. Then, the treated solution was centrifuged at 15,000 rpm for 5 minutes and filtrated using a filter to obtain the supernatant as the treated liquid.

The concentrations of an antibody as a useful protein and DNA as an impurity in the culture supernatant before and after the treatment were determined. In addition, the concentrations of a host cell protein (HCP) and histone were also determined. Specifically, the antibody concentration was analyzed by Protein A chromatography, the DNA concentration was analyzed using Host Cell DNA Amplification Kit (“Host Cell DNA Kit D555T” manufactured by Cygnus) in accordance with the accompanying protocol, the HCP concentration was analyzed using “CHO Host Cell Protein ELISA Kit, 3rd Generation” manufactured by Cygnus in accordance with the accompanying protocol, and histone H2A was analyzed by treating with trypsin and then using LC-TOFMS (combination of “UFLC Nexera X2” manufactured by SHIMADZU CORPORATION and “Triple TOF6600” manufactured by ABSCIEX), and the concentrations were respectively determined using Protein pilot and Markerview software. The result is shown in Table 3.

TABLE 3

| Additive amount of basic | IgG | DNA | HCP | Histone H2A |
|---|---|---|---|---|
| magnesium carbonate | concentration | concentration | concentration | response |
| 0 w/v % | 3.5 mg/mL | 12 ng/mL | 218 μg/mL | 631,965 |
| 1 w/v % | 3.5 mg/mL | 0 ng/mL | 135 μg/mL | 165,110 |

It was found from the result shown in Table 3 that basic magnesium carbonate can also adsorb HCP and histone to be removed in addition to DNA with maintaining IgG concentration in IgG-containing culture supernatant of an animal cell.

### Example 4: Adsorption and Removal of DNA from Culture Suspension of Animal Cell by Using Basic Magnesium Carbonate

Basic magnesium carbonate was added to a suspension of an animal cell containing monoclonal antibody (IgG) in a concentration of 1 w/v % or 3 w/v %, and the mixture was stirred at room temperature using a mix rotor for 1 hours. Then, 15 g of the treated solution was filtrated using 0.2 μm membrane filter to obtain the treated liquid. The concentrations of IgG, DNA and HCP in the obtained treated liquid were determined similarly to Example 3. The similar experiment was conducted except that basic magnesium carbonate was not added for comparison. The result is shown in Table 4.

TABLE 4

| Additive amount of | Amount of | | |
|---|---|---|---|
| basic magnesium | liquid after | IgG | DNA |
| carbonate | removal of cell | concentration | concentration |
| 0 w/v % | 4.7 g | 5.2 mg/mL | 14,450 ng/mL |
| 1 w/v % | 10.3 g | 5.5 mg/mL | 852 ng/mL |
| 3 w/v % | 14.8 g | 6.1 mg/mL | 312 ng/mL |

It was found from the result shown in Table 4 that DNA can be removed even by adding basic magnesium carbonate to a cell suspension. It was also found that basic magnesium carbonate functions as a filter aid, since clogging of a filter during filtration can be prevented in a case of larger amount of added basic magnesium carbonate.

### Example 5: Removal of Protein from Culture Supernatant Containing Protein G of E. coli by Basic Magnesium Carbonate

To a culture supernatant containing Protein G of E. coli, 1 w/v % or 10 w/v % of basic magnesium carbonate and/or 0.67 w/v % activated carbon was added, and the mixture was stirred using a mix rotor for 2 hours. Then, the mixture was centrifuged at 15,000 rpm for 5 minutes to obtain the supernatant as the treated liquid. The concentrations of Protein G were analyzed by reverse phase HPLC, and the concentrations of total proteins were analyzed by Pierce™ 660 nm Protein Assay manufactured by Thermo Scientific before and after the treatment. The concentration of an impurity protein was calculated by subtracting the Protein G concentration from the total protein concentration. The result is shown in Table 5.

TABLE 5

| Basic magnesium carbonate/ | Protein G | Impurity protein |
|---|---|---|
| activated carbon | concentration | concentration |
| No addition/No addition | 4.1 mg/mL | 12.3 mg/mL |
| 1 w % added/No addition | 4.5 mg/mL | 8.0 mg/mL |
| 10 w % added/No addition | 4.4 mg/mL | 7.3 mg/mL |
| No addition/Added | 4.2 mg/mL | 8.1 mg/mL |
| 1 w % added/Added | 3.6 mg/mL | 7.4 mg/mL |
| 10 w % added/Added | 4.2 mg/mL | 7.0 mg/mL |

It was found from the result of Table 5 that an impurity protein can be removed by using basic magnesium carbonate from a culture supernatant containing Protein G of E. coli. It was also found that an impurity protein can be further removed in combination with activated carbon, though a Protein G concentration is slightly decreased.

### Example 6: Removal of Protein from Protein G-Containing Culture Fluid of E. coli by Basic Magnesium Carbonate

To a culture supernatant of E. coli containing Protein G and a bacteria cell, 10 w/v % of basic magnesium carbonate was added. The mixture was stirred using a mix rotor for 2 hours. Then, the mixture was centrifuged at 15,000 rpm for 5 minutes to obtain the supernatant as the treated liquid. The concentrations of Protein G were analyzed before and after the treatment using Pierce™ 660 nm Protein Assay manufactured by Scientific. The impurity protein concentration was calculated by subtracting the Protein G concentration from the total protein concentration. The result is shown in Table 6.

TABLE 6

| Additive amount of basic | Protein G | Impurity protein |
|---|---|---|
| magnesium carbonate | concentration | concentration |
| 0 w/v % | 4.1 mg/mL | 12.3 mg/mL |
| 10 w/v % | 4.6 mg/mL | 9.9 mg/mL |

It was found from the result shown in Table 6 that an impurity protein can be removed from Protein G-containing culture supernatant of E. coli by basic magnesium carbonate even before a bacteria cell is broken.

### Example 7: Removal of Protein from Protein A-Containing Culture Liquid of Brevibacillus choshinensis by Basic Magnesium Carbonate

The pH of Protein A-containing Brevibacillus choshinensis culture supernatant was adjusted to 5, and 10 w/v % of basic magnesium carbonate and/or 0.67 w/v % of activated carbon was added thereto. The mixture was stirred using a mix rotor for 2 hours. Then, the mixture was centrifuged at 15,000 rpm for 5 minutes to obtain the supernatant as the treated liquid. The concentrations of Protein A were analyzed before and after the treatment by reverse phase HPLC, and the total protein concentrations were analyzed using Pierce™ 660 nm Protein Assay manufactured by Thermo Scientific. The impurity protein concentration was calculated by subtracting the Protein A concentration from the total protein concentration. The result is shown in Table 7.

TABLE 7

| Basic magnesium carbonate/ | Protein A | Impurity protein |
|---|---|---|
| activated carbon | concentration | concentration |
| No addition/No addition | 8.1 mg/mL | 14.3 mg/mL |
| Added/No addition | 7.9 mg/mL | 11.5 mg/mL |
| No addition/Added | 7.8 mg/mL | 12.6 mg/mL |
| Added/Added | 7.8 mg/mL | 10.7 mg/mL |

It was found from the result shown in Table 7 that basic magnesium carbonate has an effect to remove an impurity protein even from Protein A-containing culture supernatant of E. coli, and an excellent effect to remove an impurity protein can be obtained by the combination with activated carbon though a Protein A concentration is slightly decreased.

### Example 8: Removal of Impurity from Protein A-Containing Culture Supernatant of Blevi by Basic Magnesium Carbonate after Acid Dissociation Treatment

The pH of Protein A-containing culture supernatant of Brevibacillus choshinensis was adjusted to 5.4 using acetic acid, and the supernatant was heated at 60° C. for 60 minutes. Each sample after the treatment was centrifuged at 12,000 rpm for 5 minutes to obtain a supernatant. Basic magnesium carbonate was added to the treated culture supernatant in a concentration of 10 w/v %. The mixture was stirred using a mix rotor for 2 hours and centrifuged at 15,000 rpm for 5 minutes to obtain the supernatant as the treated liquid. The concentrations of Protein A in the culture fluid were analyzed using reverse phase HPLC, and the total protein concentrations were analyzed by Lowry method before and after the treatment. The impurity protein concentration was calculated by subtracting the Protein A concentration from the total protein concentration. The result is shown in Table 8.

TABLE 8

| Basic magnesium carbonate/ | Protein A | Impurity protein |
|---|---|---|
| activated carbon | concentration | concentration |
| No addition/No addition | 8.1 mg/mL | 1.52 mg/mL |
| Added/No addition | 7.9 mg/mL | 1.50 mg/mL |
| No addition/Added | 8.0 mg/mL | 1.38 mg/mL |
| Added/Added | 7.8 mg/mL | 0.99 mg/mL |

It was found from the result shown in Table 8 that an impurity protein can be removed even from Protein A-containing culture supernatant of E. coli subjected to an acid treatment by adding basic magnesium carbonate, and an excellent effect to remove an impurity protein can be obtained by the combination with activated carbon though a Protein A concentration is slightly decreased.

### Example 9: Adsorption and Removal of DNA by Water-Insoluble Magnesium Compound

Salmon sperm DNA solution was treated and DNA concentration was determined similarly to Example 1 except that magnesium oxide, magnesium hydroxide, magnesium silicate or magnesium phosphate was used as a water-insoluble magnesium salt. The result is shown in Table 9.

TABLE 9

| Water-insoluble magnesium compound | | |
|---|---|---|
| Compound name | Concentration | DNA concentration |
| — | — | 1.00 mg/mL |
| Granular magnesium oxide | 1 w/v % | 0.68 mg/mL |
| | 10 w/v % | 0.03 mg/mL |
| Heavy magnesium oxide | 1 w/v % | 0.75 mg/mL |
| | 10 w/v % | 0.04 mg/mL |
| Granular magnesium hydroxide | 1 w/v % | 0.67 mg/mL |
| | 10 w/v % | 0.02 mg/mL |
| Magnesium phosphate | 1 w/v % | 0.72 mg/mL |
| | 10 w/v % | 0.03 mg/mL |
| Magnesium silicate | 1 w/v % | 0.75 mg/mL |
| | 10 w/v % | 0.02 mg/mL |

It was found from the result shown in Table 9 that magnesium oxide, magnesium hydroxide, magnesium silicate and magnesium phosphate as the water-insoluble magnesium salt also have an effect to remove DNA.

### Example 10: Adsorption and Removal of DNA from Insulin Solution by Basic Magnesium Carbonate

To 0.1 mg/mL insulin solution, 1 w/v % of basic magnesium carbonate was added similarly to Example 1. The mixture was stirred using a mix rotor for 1 hour. Then, the mixture was centrifuged at 15,000 rpm for 5 minutes to obtain the supernatant as the treated liquid. The insulin recovery rate was determined by dividing the absorbance at UV 280 nm of the treated solution by the absorbance at UV 280 nm of the solution before the treatment.

As a result, the recovery rate of insulin was 90% and thus it was found that insulin can be recovered with high yield by basic magnesium carbonate. It was also found that DNA can be efficiently removed by the same condition as the result of Example 1.

### Example 11: Adsorption and Removal of DNA from Protease Solution by Basic Magnesium Carbonate

To 1 mg/mL protease aqueous solution, 1 w/v % of basic magnesium carbonate was added similarly to Example 1. The mixture was stirred using a mix rotor for 1 hour. Then, the mixture was centrifuged at 15,000 rpm for 5 minutes to obtain the supernatant as the treated liquid. The protease recovery rate was determined by dividing the absorbance at UV 280 nm of the treated solution by the absorbance at UV 280 nm of the solution before the treatment.

As a result, the recovery rate of protease was 94% and thus it was found that protease can be recovered with high yield by basic magnesium carbonate. It was also found that DNA can be efficiently removed by the same condition as the result of Example 1.

### Example 12: Adsorption and Removal of DNA from Epidermal Growth Factor (EGF) Solution by Basic Magnesium Carbonate

To 10 ng/mL epidermal growth factor aqueous solution, 1 w/v % of basic magnesium carbonate was added similarly to Example 1. The mixture was stirred using a mix rotor for 1 hour. Then, the mixture was centrifuged at 15,000 rpm for 5 minutes to obtain the supernatant as the treated liquid. The concentrations of EGF in the treated solution and the solution before the treatment were determined using Human EGF Quantikine ELISA kit manufactured by R&D Systems, and EGF recovery rate was calculated by dividing the EGF concentration after the treatment by the EGF concentration before the treatment.

As a result, the recovery rate of EGF was 94% and thus it was found that EGF can be recovered with high yield by basic magnesium carbonate. It was also found that DNA can be efficiently removed by the same condition as the result of Example 1.

### Example 13: Adsorption and Removal of DNA from Human Serum Albumin Solution by Basic Magnesium Carbonate

To 1 mg/mL human serum albumin aqueous solution, 1 w/v % of basic magnesium carbonate was added similarly to Example 1. The mixture was stirred using a mix rotor for 1 hour. Then, the mixture was centrifuged at 15,000 rpm for 5 minutes to obtain the supernatant as the treated liquid. The human serum albumin recovery rate was determined by dividing the absorbance at UV 280 nm of the treated solution by the absorbance at UV 280 nm of the solution before the treatment.

As a result, the recovery rate of human serum albumin was 90% and thus it was found that human serum albumin can be recovered with high yield by basic magnesium carbonate.

### Example 14: Adsorption and Removal of Nucleic Acid by Basic Magnesium Carbonate

Removal rates of plasmid and ribosome were determined similarly to Example 1 except that 1 μg/mL aqueous solution of plasmid as cyclic DNA, 1 μg/mL aqueous solution of a DNA fragment or 1 μg/mL aqueous solution of ribosome as a complex of nucleic acid and a protein was treated. The result is shown in Table 10.

TABLE 10

| Nucleic acid | Sample name | Removal rate |
|---|---|---|
| Cyclic DNA | Plasmid DNA | 98% |
| DNA fragment (500-100 bp) | DNA derived from | 81% |
| | salmon sperm | |
| RNA-protein complex | E. coli Ribosome | 99% |

It was found from the result shown in Table 10 that plasmid as cyclic DNA, a DNA fragment and ribosome as a complex of nucleic acid and a protein can be also removed at a high rate by basic magnesium carbonate.

### Example 15: Improvement of Filterability of CHO Cell Suspension by Water-Insoluble Magnesium Compound

Basic magnesium carbonate, magnesium hydroxide or magnesium oxide was added to 400 μL of CHO cell suspension in a concentration of 10 wt %. The mixture was stirred and then centrifuged at 9000 G for 1 minute using a spin column. The filtrate yield was determined by dividing the obtained filtrate by the added cell suspension amount. As a result, an insoluble component such as a cell and the water-insoluble magnesium compound could be separated by filtration without clogging. The filtrate yield is shown in FIG. 1.

It was found from the result shown in FIG. 1 that the filtrate yield from CHO cell suspension can be improved about 6 times by adding the water-insoluble magnesium compound.

### Example 16: Improvement of Filterability of HEK Cell Suspension by Water-Insoluble Magnesium Compound

To 400 μL of HEK cell suspension, basic magnesium carbonate, magnesium hydroxide or magnesium oxide was added in a concentration of 10 wt %. The mixture was stirred and then centrifuged at 9000 G for 1 minute using a spin column. The filtrate yield was determined by dividing the obtained filtrate by the added cell suspension amount. As a result, an insoluble component such as a cell and the water-insoluble magnesium compound could be separated by filtration without clogging. The filtrate yield is shown in FIG. 2.

It was found from the result shown in FIG. 2 that the filtrate yield from HEK cell suspension can be improved about 14 times by adding the water-insoluble magnesium compound.

### Example 17: Production of Layered Basic Magnesium Carbonate-Filled Device

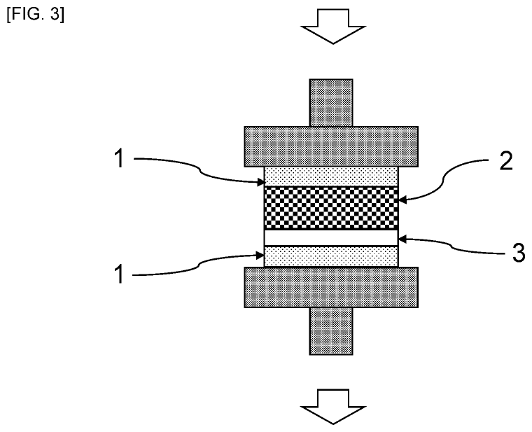

One PTFE filter having a thickness of about 1 mm manufactured by Diba was put on the bottom part of a filter holder having an inner diameter of 15 mm and a bed height of 4.5 mm, and a glass fiber depth filter having a thickness of about 0.5 mm manufactured by Millipore was put thereon. The filter holder was filled with basic magnesium carbonate by adding a suspension in which 200 mg of basic magnesium carbonate manufactured by KISHIDA CHEMICAL was dispersed in ultrapure water thereon and pulling out the ultrapure water from the bottom of the filter holder using a syringe barrel. The same PTFE filter manufactured by Diba was put on the formed basic magnesium carbonate layer to produce a layered basic magnesium carbonate-filled device. A schematic diagram of the layered device filled with basic magnesium carbonate produced in Example 17 is shown as FIG. 3. The volume of the basic magnesium carbonate for filling was 0.35 mL.

### Example 18: Production of Distributed Basic Magnesium Carbonate-Filled Device

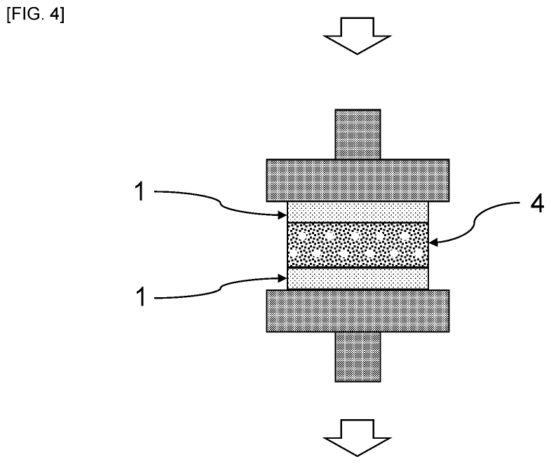

One PTFE filter having a thickness of about 1 mm manufactured by Diba was put on the bottom part of a filter holder having an inner diameter of 15 mm and a bed height of 4.5 mm. The glass fiber of a glass fiber depth filter having a thickness of about 0.5 mm manufactured by Millipore was sleaved. In ultrapure water, 20 mg of the sleaved glass fiber and 200 mg of basic magnesium carbonate manufactured by KISHIDA CHEMICAL were dispersed. The filter holder was filled with the obtained suspension thereon. The ultrapure water was taken out from the bottom of the filter holder using a syringe barrel. The same PTFE filter manufactured by Diba was put on the thus formed glass fiber-basic magnesium carbonate mixed layer to produce a distributed basic magnesium carbonate-filled device. A schematic diagram of the distributed basic magnesium carbonate-filled device produced in Example 18 is shown as FIG. 4. The volume of the basic magnesium carbonate for filling in the basic magnesium carbonate-containing layer was 0.35 mL.

### Comparative Example 1: Production of Distributed Hydrotalcite-Filled Device

One PTFE filter having a thickness of about 1 mm manufactured by Diba was put on the bottom part of a filter holder having an inner diameter of 15 mm and a bed height of 4.5 mm. The glass fiber of a glass fiber depth filter having a thickness of about 0.5 mm manufactured by Millipore was sleaved. In ultrapure water, 20 mg of the sleaved glass fiber and 200 mg of hydrotalcite manufactured by Wako were dispersed. The filter holder was filled with the obtained suspension thereon. The ultrapure water was taken out from the bottom of the filter holder using a syringe barrel. The same PTFE filter manufactured by Diba was put on the thus formed glass fiber-hydrotalcite mixed layer to produce a distributed hydrotalcite-filled device. The volume of the hydrotalcite for filling in the hydrotalcite-containing layer was 0.35 mL.

### Comparative Example 2: Production of Control Device without Filler

One PTFE filter having a thickness of about 1 mm manufactured by Diba was put on the bottom part of a filter holder having an inner diameter of 15 mm and a bed height of 4.5 mm, and a glass fiber depth filter having a thickness of about 0.5 mm manufactured by Millipore was put thereon. One of the same PTFE filter manufactured by Diba was further put thereon to produce a control device.

### Test Example 1: Evaluation of Capability to Remove Impurity of Each Device

Each device of Examples 17 and 18 and Comparative examples 1 and 2 was connected to a chromatography system (“AKTA Avant25” manufactured by GE Healthcare) to evaluate the capability to remove an impurity. Specifically, CHO culture fluid containing a monoclonal antibody (IgG) (20 mL) was supplied. The weight of the supplied culture fluid corresponded to about 100 times of the weight of the basic magnesium carbonate filling the devices of Examples 17 and 18. The velocity of the supplied liquid was set at 0.35 mL/min so that the residence time of the liquid in the basic magnesium carbonate filling the devices of Examples 17 and 18 and the hydrotalcite filling the device of Comparative example 1 became 1 minute. The culture fluid that passed through the device was collected in increments of 1.7 mL using a fraction collector of AKTA Avant25. The amounts of IgG and an impurity in the last fraction were measured by the following method.

The IgG concentration was measured using a general chromatography system including Protein A affinity column (“TSKgel Protein A-5PW” manufactured by Tosoh).

The content amount of host cell protein (HCP) was measured using a kit to detect host cell protein (“CHO Host Cell Protein ELISA Kit, 3rd Generation” manufactured by Cygnus) in accordance with the accompanying protocol.

The content amount of DNA was measured using a kit to detect DNA derived from host cell (“CHO DNA Amplification Kit in Tubes” manufactured by Cygnus) in accordance with the accompanying protocol. The result is shown in Table 11.

TABLE 11

| | | IgG | HCP | DNA |
|---|---|---|---|---|
| | Filler | recovery rate | concentration | concentration |
| Example 17 | Basic magnesium | 99% | 165 μg/mL | 0.03 ng/mL |
| | carbonate (layered) | | | |
| Example 18 | Basic magnesium | 100% | 154 μg/mL | 0.02 ng/mL |
| | carbonate (dispersed) | | | |
| Comparative | Hydrotalcite (dispersed) | 92% | 166 μg/mL | 0.06 ng/mL |
| example 1 | | | | |
| Comparative | No addition | 100% | 177 μg/mL | 0.45 ng/mL |
| example 2 | | | | |

It was demonstrated from the result shown in Table 11 that an impurity protein and DNA derived from a host cell are hardly removed by only a general depth filter (Comparative example 2).

When hydrotalcite, which is used as an adsorbent, was used in addition to a depth filter (Comparative example 1), an impurity protein and DNA derived from a host cell could be relatively removed but IgG recovery rate was decreased since hydrotalcite might also adsorb IgG as the target.

On the one hand, it was demonstrated that when basic magnesium carbonate was used in addition to a depth filter, an amount of adsorbed IgG was small but an impurity protein and DNA derived from a host cell could be adsorbed and removed more efficiently than hydrotalcite in both cases where the layer consisting of basic magnesium carbonate was used (Example 17) and basic magnesium carbonate was dispersed in the glass fiber derived from a depth filter (Example 18).

### Test Example 2: Comparison with Commercially Available Depth Filter

The filter device produced in Example 17, Example 18 or Comparative example 2 or a commercially available depth filter (“Millistak A1HC” manufactured by Merck Millipore) (Comparative example 3) was connected to a rotary pump, and 100 mg/L salmon sperm DNA solution was supplied at a velocity of 1 mL/min and fractions passing through the device were collected. The DNA removal rate was calculated by measuring the absorbance at UV 260 nm before and after the solution was supplied. The result is shown in Table 12.

TABLE 12

| | | DNA |
|---|---|---|
| | Filler | concentration |
| Example 17 | Basic magnesium carbonate | 65% |
| | (layered) | |
| Example 18 | Basic magnesium carbonate | 66% |
| | (dispersed) | |
| Comparative | No addition | 3% |
| example 2 | | |
| Comparative | Commercially available depth | 10% |
| example 3 | filter | |

It was demonstrated from the result shown in Table 12 that DNA is difficult to remove by only a general depth filter (Comparative example 2) or a commercially available depth filter (Comparative example 3), which contain a water-insoluble medium and which do not contain a filler.

On the one hand, it was demonstrated that DNA as an impurity can be efficiently adsorbed and removed by the filter device of the present invention having the layer containing basic magnesium carbonate.

### Test Example 3: Evaluation of Antibody Recovery Rate by Filter Device

The fractions that passed through the device were collected similarly to Test example 1 except that 25 mM tris-HCl buffer (pH 7.5) containing 1 g/L human blood-derived polyclonal antibody (IgG) and 500 mM sodium chloride was used in place of the culture supernatant. The IgG recovery rate was determined by measuring the absorbance at UV 280 nm before and after the buffer was passed through the device. The result is shown in Table 13.

TABLE 13

| | Filler | IgG recovery rate |
|---|---|---|
| Example 18 | Basic magnesium carbonate | 85% |
| | (dispersed) | |
| Comparative | Hydrotalcite (dispersed) | 19% |
| example 1 | | |

As the result shown in Table 13, when hydrotalcite, which is also used as an adsorbent, was used (Comparative example 1), the IgG recovery rate was remarkably decreased. This result may be because hydrotalcite adsorbs IgG.

On the one hand, when the filter device of the present invention containing the basic magnesium carbonate-containing layer, IgG could be successively recovered.

### Test Example 4: Evaluation of Antibody Recovery Rate by Filter Device

Sodium chloride was added to 25 mM tris-HCl buffer of pH 7.5 in a salt concentration of 0 mM, 100 mM, 500 mM or 1000 mM. Human blood-derived polyclonal antibody (IgG) was added to the buffer in a concentration of 1 g/L. The mixture was stirred well. Basic magnesium carbonate or hydrotalcite was added to the thus obtained solution in a concentration of 1 wt %. The mixture was stirred for 1 hour, and then centrifuged. The IgG recovery rate was determined by the absorbance at UV 280 nm of the solution. The result is shown in Table 14.

TABLE 14

| | Antibody recovery rate | | | |
|---|---|---|---|---|
| Additive | 0 mM | 100 mM | 500 mM | 1,000 mM |
| Basic magnesium carbonate | 81% | 86% | 92% | 95% |
| Hydrotalcite | 18% | 23% | 33% | 46% |

It was found from the result shown in Table 14 that the antibody recovery rate becomes lower in the condition of a lower salt concentration, and the antibody recovery rate by basic magnesium carbonate is much higher than the antibody recovery rate by hydrotalcite.

### Example 19: Velocity of Liquid Passing Through Layered Basic Magnesium Carbonate-Filled Device

One PTFE filter having a thickness of about 1 mm manufactured by Diba was put on the bottom part of a filter holder having an inner diameter of 7 mm and a bed height of 25 mm. The glass fiber of a glass fiber depth filter having a thickness of about 0.5 mm manufactured by Millipore was sleaved. In ultrapure water, 20 mg of the sleaved glass fiber and 1000 mg of basic magnesium carbonate manufactured by KISHIDA CHEMICAL were dispersed. The filter holder was filled with the obtained suspension thereon. The ultrapure water was taken out from the bottom of the filter holder using a syringe barrel. The same PTFE filter manufactured by Diba was put on the thus formed glass fiber-basic magnesium carbonate mixed layer to produce a distributed basic magnesium carbonate-filled device. The volume of the basic magnesium carbonate for filling in the basic magnesium carbonate-containing layer was 1 mL.

In addition, a hydrotalcite-filled column was similarly prepared as Comparative example 4 except that hydrotalcite was used in place of basic magnesium carbonate.

The column prepared as the above was connected to a chromatography system (“AKTA Avant25” manufactured by GE Healthcare), 100 mg/L salmon sperm DNA solution was supplied thereto at a velocity of 0.1 mL/min (15.6 cm/hr), 1 mL/min (156 cm/hr) or 10 mL/min (1560 cm/hr), and the fractions that passed through the device were collected. The DNA removal rates were determined by measuring the absorbance at UV 260 nm before and after the solution was passed through the device. The result is shown in Table 15.

TABLE 15

| | DNA removal rate | | |
|---|---|---|---|
| Filler | 0.1 mL/min | 1 mL/min | 10 mL/min |
| Basic magnesium carbonate | 61% | 68% | 65% |
| No addition | 2% | 1% | 2% |

It was found from the result shown in Table 15 that DNA as an impurity can be effectively removed by using the filter device of the present invention having the basic magnesium carbonate-containing layer in various velocities of passing liquid.

### Example 20: Layered Magnesium Hydroxide-Filled Device

The DNA removal rate was determined using each device similarly to Example 19 except that magnesium phosphate (1000 mg) or magnesium hydroxide (1000 mg) was used in place of basic magnesium carbonate (1000 mg) and the velocity was adjusted to 1 mL/min (156 cm/hr). The result is shown in Table 16.

TABLE 16

| Filler | DNA removal rate |
|---|---|
| Basic magnesium carbonate (dispersed) | 60% |
| Basic magnesium hydroxide (dispersed) | 36% |
| Basic magnesium phosphate (dispersed) | 41% |
| No addition | 1% |

It was demonstrated as the result shown in Table 16 that DNA as an impurity can be effectively reduced even in the case where magnesium phosphate and magnesium hydroxide are used.

### Example 21: Water-Insoluble Medium Used for Device

The DNA removal rate was determined using each device similarly to Example 19 except that agarose, cellulose, cellulose acetate, activated carbon, diatomite, pearlite, hydrotalcite, milled fiber, glass fiber, alumina, silica gel, zirconia, barium titanate, polyacrylonitrile, polyester, polyether sulfone, polypropylene or PTFE was used as a water-insoluble medium. The result is shown in Table 17.

TABLE 17

| Support | DNA removal rate |
|---|---|
| Cellulose | 69.0% |
| Cellulose acetate | 67.0% |
| Agarose ABT | 63.3% |
| Activated carbon | 86.3% |
| Hydrotalcite | 70.7% |
| Pearlite | 52.8% |
| Diatomite | 62.1% |
| Milled fiber | 57.8% |
| Polyacrylonitrile | 57.2% |
| Polyester | 65.0% |
| PES (Msutang S) | 60.7% |
| Polypropylene | 64.8% |
| PTFE | 63.1% |
| Glass fiber | 64.1% |
| Alumina | 69.6% |
| Silica gel | 71.1% |
| Zirconia | 78.7% |
| Barium titanate | 63.0% |

It was demonstrated as the result shown in Table 17 that DNA can be effectively removed by using the filter device of the present invention having the basic magnesium carbonate-containing layer even when agarose, cellulose, cellulose acetate, activated carbon, diatomite, pearlite, hydrotalcite, milled fiber, glass fiber, alumina, silica gel, zirconia, barium titanate, polyacrylonitrile, polyester, polyether sulfone, polypropylene and PTFE are used as a water-insoluble medium.

### REFERENCE SIGNS LIST

1: PTFE filter
2: Water-insoluble magnesium compound-containing layer consisting of water-insoluble magnesium compound
3: Depth filter
4: Water-insoluble magnesium compound-containing layer containing water-insoluble magnesium compound particle and water-insoluble medium

1. A method for reducing an amount of a nucleic acid in a liquid, the method comprising the steps of:
contacting the liquid with a water-insoluble magnesium compound to adsorb at least a part of the nucleic acid on the water-insoluble magnesium compound, and then
separating the liquid from the water-insoluble magnesium compound.

2. The method according to claim 1, wherein the water-insoluble magnesium compound comprises one or more selected from magnesium carbonate, magnesium hydroxide, magnesium oxide, magnesium silicate and magnesium phosphate.

3. The method according to claim 1, wherein the liquid further comprises a useful substance.

4. The method according to claim 1, wherein the liquid is separated from the water-insoluble magnesium compound by filtration.

5. The method according to claim 3, wherein the useful substance is one or more useful substances selected from a useful protein, a virus and a virus-like particle.

6. The method according to claim 5, wherein the useful protein is one or more useful proteins selected from an antibody, an antibody-like molecule, a hormone, an enzyme, a growth factor, a blood protein and an antibody-binding protein.

7. The method according to claim 1, wherein the liquid is a cell culture fluid or a body fluid.

8. An adsorbing filter comprising a layer comprising a water-insoluble magnesium compound.

9. The adsorbing filter according to claim 8, comprising the layer consisting of a water-insoluble magnesium compound particle.

10. The adsorbing filter according to claim 8, comprising the layer comprising the water-insoluble magnesium compound and a water-insoluble medium.

11. The adsorbing filter according to claim 10, wherein a material of the water-insoluble medium comprises one or more selected from a polysaccharide, a synthetic polymer and an inorganic substance.

12. The adsorbing filter according to claim 11, wherein the polysaccharide comprises one or more selected from cellulose, cellulose acetate, nitrocellulose, agarose and chitosan.

13. The adsorbing filter according to claim 11, wherein the synthetic polymer comprises one or more selected from polyacrylonitrile, polyester, polyether sulfone, polypropylene and polytetrafluoroethylene.

14. The adsorbing filter according to claim 11, wherein the inorganic substance comprises one or more selected from glass, silica, alumina, zirconia and barium titanate.

15. The adsorbing filter according to claim 8, wherein the water-insoluble magnesium compound comprises one or more selected from magnesium carbonate, magnesium hydroxide, magnesium oxide and magnesium phosphate.

16. A method of producing a liquid having a reduced amount of nucleic acid, the method comprising the steps of:
contacting the liquid containing an amount of nucleic acid with a water-insoluble magnesium compound to adsorb at least a part of the nucleic acid on the water-insoluble magnesium compound, and then
separating the liquid from the water-insoluble magnesium compound to produce said liquid having a reduced amount of nucleic acid.

17. A method of producing a useful substance by reducing an amount of nucleic acid in a liquid medium, the method comprising the steps of:
contacting the liquid containing a useful substance and nucleic acid with a water-insoluble magnesium compound to adsorb at least a part of the nucleic acid on the water-insoluble magnesium compound, and then
separating the liquid from the water-insoluble magnesium compound to produce said liquid having a reduced amount of nucleic acid.

18. The method according to claim 17, further comprising the water-insoluble magnesium compound comprises one or more selected from magnesium carbonate, magnesium hydroxide, magnesium oxide, magnesium silicate and magnesium phosphate.

19. The method according to claim 17, wherein the useful substance is one or more useful substances selected from a useful protein, a virus and a virus-like particle.

20. The method according to claim 19, wherein the useful protein is one or more useful proteins selected from an antibody, an antibody-like molecule, a hormone, an enzyme, a growth factor, a blood protein and an antibody-binding protein.
